(19) United States
(12) Patent Application Publication
(10) Pub. No.: US 2024/0238190 A1
(43) Pub. Date: Jul. 18, 2024

(54) COSMETIC COMPOSITIONS COMPRISING A BARBARY FIG EXTRACT FOR MOISTURISING KERATIN FIBRES

(71) Applicant: PIERRE FABRE DERMO-COSMETIQUE, LAVAUR (FR)

(72) Inventors: Sylvie DAUNES-MARION, CASTRES (FR); Cédric FRIBAULT, CASTRES (FR); David BLACK, CASTRES (FR); Stéphane POIGNY, CASTRES (FR)

(73) Assignee: PIERRE FABRE DERMO-COSMETIQUE, LAVAUR (FR)

(21) Appl. No.: 18/562,079

(22) PCT Filed: May 20, 2022
(86) PCT No.: PCT/FR2022/050967

§ 371 Date: Nov. 17, 2023

(30) Foreign Application Priority Data
May 20, 2021 (FR) FR2105306

(51) Int. Cl. A61K 8/9789; A61Q 5/00

(52) U.S. Cl. CPC A61K 8/9789; A61Q 5/002

(57) ABSTRACT

The present invention relates to the use of a Barbary fig extract for tightening the scales of keratin fibres.

### TECHNICAL FIELD OF THE INVENTION

The field of the invention is the care, in particular the moisturizing of skin appendages, more particularly keratin fibers.

The invention relates in particular to compositions comprising a prickly pear extract in a physiologically acceptable medium suitable for topical application to the skin appendages.

It also relates to a cosmetic care method, in particular for moisturizing the skin appendages, using said compositions.

### STATE OF THE ART

Keratins are fundamental components of skin, hair, eyelashes and nails. Hair is composed of 95% keratin, a fibrous and helical protein. Keratin, synthesized by keratinocytes, is insoluble in water, ensuring hair's impermeability and protection. The hair structure is divided into three distinct parts:
Medulla, the central part of the shaft, composed of an amorphous, soft, oily substance;
Cuticle, a thin, protective outer layer that contains the nourishing substance essential to hair growth. This highly keratinized layer is made up of scale-like cells that overlap one another;
Cortex, the main component of the hair, containing long keratin chains that add elasticity, suppleness and strength to the hair. The cells of the cortex are joined together by an intercellular cement rich in lipids and proteins. Each cell is composed of bundles that lie in the direction of the hair length, known as macrofibrils, which in turn are made up of microfibrils, which in turn are made up of protofibrils.

Keratin has a certain affinity for water. At saturation, hair can absorb up to 30% to 40% of its weight in water. Absorption depends on the relative humidity of the surrounding environment, and affects the hair shaping. In fact, the physical properties of keratin fibers are modified by the presence of water, such as variations in length, diameter and internal viscosity.

Hair is permeable to water in both vapor and liquid form. Water bonds to keratin by creating hydrogen bonds. Water absorption through the cuticle and cortex causes hair swelling rather than lengthening.

Swelling is strongly influenced by pH, which is reduced at very acidic pH and increased at very basic pH. Thermal balance is also a factor, with a rise in temperature accelerating water penetration and therefore capillary moisture.

The cuticular surface, which is relatively permeable to small molecules from the external environment, is described as porous. When intact, it acts as a barrier, but only for macromolecules and not for water.

Dry hair is characterized by its overall dull appearance and rough feel. Dry, dehydrated hair is characterized by a dull, brittle, frothy appearance that swells easily on contact with humidity. Its overall appearance appears unstructured, and it is often difficult to style dry hair. Dry hair results from insufficient sebum production by the sebaceous glands and a lack of water in the hydrolipidic film. The latter protects the scalp and spreads over the hair, helping to lubricate it. Dryness and dehydration of the scalp and hair prevent protection and lubrication. As a result, the hair shaft undergoes damage, first to the cuticle and then to the interior of the fiber. This deterioration of the hair shaft is seen in a change in the shape and position of the scales, which are lifted; a gradual disappearance of the cuticle; and damage to the cortical keratin, which loses its elasticity.

Hair may be dry constitutionally, due to insufficient sebaceous secretion, or it may have been dried out as a result of various aggressions. This phenomenon increases with age.

Constitutional causes of dry hair include hereditary conditions such as woolly hair, trichothiodystrophy, hairless hair syndrome, hereditary frizzy hair and trichorrhexia nodosa. Other endogenous causes of dry hair include thyroid dysfunction, such as hypothyroidism or pituitary insufficiency, which can cause hyposeborrhea; and menopause, which can lead to brittle, dry hair due to reduced sebaceous secretion.

Finally, medications such as isotretinoin and cyproterone acetate can cause hyposebborrhea.

But the main causes are exogenous: hair can become dry as a result of various aggressions.

Aggressive environmental conditions such as rain, wind, sun, sudden variations in temperature and humidity, sea salt, chlorine or salt from swimming pools, pollution and tobacco can damage the structure of skin appendages, especially keratin fibers, particularly hair. Aggressive chemical treatments, such as shampoos, cosmetics for make-up, coloring, bleaching, perming and/or styling, and physical treatments such as heat from hair dryers and straightening irons, and friction from brushing, also damage keratin fibers, and hair in particular. Biological aggressors such as bacteria and fungi can also cause oxidative stress, which can damage the skin appendages.

Nails are also subject to environmental aggressors such as cold, chemical aggressors such as varnishes, solvents and household products, biological aggressors such as fungi, and mechanical aggressors such as friction.

These conditions and/or aggressive agents alter keratin fibers, particularly at cuticle level, to the point of lifting the scales. Keratin fibers are then weakened. Over time and/or depending on how often and/or how intensely they are exposed to aggression, the scales detach from the surface of the keratin fibers. When damaged, they lose their structure and/or visual and/or biomechanical properties. As a result, damaged hair in particular is dry, rough and therefore less soft, difficult to style and shape compared to undamaged hair, and is brittle, particularly split tips. This loss of hair surface quality is visible and unsightly. Hair also reflects less light, leaving it visibly less shiny and less bright.

On normal hair, i.e., non-dry hair, a reduction in the number of cuticular scale layers is observed from the root to the tip. These scales, uniform in appearance at the root, become eroded at the tips, with saw-toothed edges, and detach from the shaft. In long hair, the cuticle frequently disappears at the tips, exposing the cortex. The hair then suffers from swallowtail fractures, more commonly known as split tips.

Cosmetic approaches therefore consist in protecting the structure of keratin fibers by moisturizing them to restore their integrity and/or their visual properties such as their softness and/or silkiness, their shine and/or their lustre, and/or their biomechanical properties such as their resistance, vitality and/or strength, and in particular for hair, to facilitate styling and/or shaping.

Numerous ingredients designed to protect and moisturize keratin fibers, particularly hair, against aggression already exist in cosmetics, but there is a constant need for alternatives to overcome formulation constraints and offer the cosmetics market a variety of solutions in terms of mode of action and effectiveness.

### SUMMARY OF THE INVENTION

The present invention relates to the cosmetic use of a prickly pear extract to tighten the scales of keratin fibers.

The present invention also relates to the non-therapeutic use of a cosmetic composition comprising a prickly pear extract and at least one cosmetically acceptable excipient, for tightening the scales of keratin fibers.

Other aspects of the invention are as described in the claims and below.

### Definitions

The term “skin appendages” refers to integumentary products derived from the ectoderm and characterized by a high level of keratinization, in particular keratin fibers. Skin appendages therefore do not include skin or mucous membranes. In humans, the main skin appendages are hair and nails.

For the purposes of this invention, the term “keratin fibers” means hair, eyelashes, eyebrows and body hair, preferably beard hair. According to the invention, the beard includes the moustache.

For the purposes of the present invention, “protecting skin appendages” means at least partially maintaining the structure and/or visual properties and/or biomechanical properties of skin appendages, preferably keratin fibers, even more preferably hair. This protection is preferably assessed when skin appendages are exposed to environmental, chemical, biological and/or mechanical aggression.

Advantageously, the protection of keratin fibers, in particular hair, involves protecting these fibers when they are subjected to physical or mechanical aggression, alteration, degradation, damage or stress. In particular, these alterations, degradations, damage or mechanical stresses can be chosen from the group comprising brushing, straightening, drying, pulling, combing and curling, either individually or in combination.

Equally advantageously, the protection of keratin fibers, in particular hair, concerns the protection of these fibers when they are subjected to chemical aggressions selected from dyeing and/or bleaching.

Environmental aggressions include fumes, pollution, temperature (especially hot and cold and their sudden variations), water content (especially humidity or dryness), solar irradiation (especially visible spectra, UV and/or gamma rays), rain, wind, dust and sea salt.

Chemical aggressions include aggressive household products, chlorine from swimming pools, certain cosmetic products which can be aggressive, such as colorants and bleaches, varnishes and/or solvents, make-up products. Chemical aggressions to the hair include, in particular, aggressive shampoos and hair care and/or treatment products, notably for styling, shaping such as straightening and/or penning and/or for coloring and/or bleaching.

Physical aggressions include friction such as brushing and combing and/or rubbing against fabrics and/or particles such as dust and/or sand, heat such as hair dryers and straightening irons, and/or styling such as exposure to pulling, stretching and/or twisting forces.

Biological aggressions on the skin appendages are mainly of a microbiological nature. According to the invention, biological aggression refers to microorganisms that damage the structure and/or visual and/or biomechanical properties of dander, in particular bacteria and/or fungi.

Bacteria and/or fungi can contribute to oxidative stress, altering the structure and/or properties of the skin appendages. In the case of hair, this oxidative stress occurs via the oxidation of scalp lipids present in the infundibular canal where sebum produced by the sebaceous gland flows. Fungi, particularly myoses, alter the structure and quality of nails, making them dry, brittle, yellow and stained.

In the present invention, “cosmetically acceptable” means that which is useful in the preparation of a cosmetic composition, which is generally safe, non-toxic and neither biologically nor otherwise undesirable, and which is acceptable for cosmetic use particularly by topical application. “Topical application” means application to the skin, mucous membranes and/or skin appendages.

For the purposes of this invention, “hydrophilic solvent” means a solvent selected from, for example, water, subcritical water, water-miscible alcohols such as ethanol, C3 to C5 glycols, glycerol, acetone and mixtures thereof.

For the purposes of the present invention, “dry extract” means an extract that contains no extraction solvent or carrier, or only insignificant traces thereof. Such a dry extract thus contains only material from the prickly pear. It may also contain insignificant traces of extraction solvent.

### DETAILED DESCRIPTION OF THE INVENTION

The purpose of the present invention is to meet the needs previously expressed. The inventors unexpectedly discovered that a prickly pear extract had the ability to protect keratin fibers, in particular hair. Prickly pear extract has the advantage of acting directly on keratin fibers, moisturizing them and tightening the scales. By tightening the scales, prickly pear extract has a protective, repairing effect on the surface of the hair fiber. This protective effect is evident not only on normal hair, but also on damaged hair.

A first object of the invention thus relates to the cosmetic use of a prickly pear extract to tighten the scales of keratin fibers, advantageously hair.

A second object of the invention relates to the non-therapeutic use of a cosmetic composition comprising at least one prickly pear extract with at least one cosmetically acceptable excipient, to tighten the scales of keratin fibers, advantageously hair.

The prickly pear extract or the composition comprising such an extract thus makes it possible to protect and/or repair keratin fibers.

Prickly pear extract useful in the context of the present invention may be as described below.

Prickly Pear Extract

In the present invention, “prickly pear extract” is taken to mean an extract of all or part of the Opuntia ficus-indica plant obtained by a solid/liquid extraction method.

For the purposes of the present invention, “extract” means the product obtained after solid/liquid extraction of all or part of the plant, in particular the stem called cladode for plants of the Cactaceae family, with at least one solvent, called extraction solvent, i.e., a product containing all the soluble compounds present in the extraction solvent may then optionally be in concentrated, standardized or dry form after partial or total evaporation of the extraction solvent. The extract useful in the context of the invention may be in the form of a dry extract, advantageously in powder form.

The extract according to the invention can be a standardized extract via the addition of a carrier or one or more excipients. The prickly pear is a fat plant belonging to the cactus family, and more specifically to the Opuntia genus. It is grown in arid climates such as the Mediterranean and Central America. The semi-arid regions of Mexico contain the greatest diversity of cacti in the world. The Opuntia genus contains around 300 species, many of which produce edible stems and fruit. These include Opuntia ficus-indica (thornless) and Opuntia megacantha (thorny).

According to the present invention, the prickly pear is Opuntia ficus-indica.

The prickly pear is an arborescent plant with flat, oval, fleshy, snowshoe-shaped stems that can reach 3 to 4 meters in height. The stems, called cladodes, are 30 to 40 cm long, 15 to 25 cm wide and 1.5 to 3 cm thick. Green in color, they join together to form a kind of branch. They are covered with a waxy cuticle that limits the plant's transpiration. The prickly pear produces flowers and fruit in abundance. The flowers appear on top of the cladodes, and are edible, as are the fruits they give rise to, which take the form of large, fleshy, ovoid berries.

Young cladodes are eaten as a vegetable, as they are tender and fibrous. Their nutritional value is similar to that of many leafy vegetables. They are rich in water, carbohydrates, protein, vitamin C and b-carotene. The chemical composition of cladodes depends on variety, growth stage and environmental conditions. Recent studies have shown that the mineral composition is high, with mainly potassium, calcium and magnesium, and to a lesser extent manganese, iron and zinc. Prickly pear cladodes are rich in polyphenols, mainly flavonoids and phenolic acids (El-Mostapha et al. Molecules 2014, 19 (9), 14879-14901).

The whole plant or the fruit and/or shoots of prickly pear are typically extracted using a maceration method with an aqueous solvent or solvent mixture. In non-limiting cases, extracts can be obtained by extraction methods in which the plant, in particular plant parts such as cladodes, are macerated and then exposed to an extraction solvent. Insoluble materials and particles can be filtered to obtain the liquid phase. The extractant may be water or alcohol, or a combination of these (i.e., a hydroalcoholic mixture). The alcohol can be methanol, ethanol, glycerol or a glycol (e.g., ethylene glycol). In particular, the extraction solvent is water or a water/glycerol mixture in a v/v ratio of between 40/60 and 60/40.

Aqueous, alcoholic and hydroalcoholic extracts can include ingredients that are soluble in the extraction agent used. For example, an aqueous extract may include an alcohol such as glycerol or 2-phenoxyethanol.

In the context of the present invention, prickly pear extract is obtained via a solid/liquid extraction method, in particular obtained from the stems, or cladodes, of the prickly pear plant, Opuntia ficus-indica. Typically, enzymatic hydrolysis of the stems, or cladodes, is carried out prior to solid/liquid extraction.

Prickly pear cladodes can be fresh or dry, whole, cut or ground, then subjected to an enzymatic hydrolysis step followed by an extraction step. Advantageously, prickly pear cladodes are reduced to powder.

A method of preparing an extract useful in the context of the invention comprises a step of extracting prickly pear plant cladodes with a hydrophilic solvent, preferably water, or even a hydroalcoholic solvent, in particular a water/glycerol mixture.

In a preferred embodiment of the invention, the extraction solvent can be chosen from water or a water/glycerol mixture, advantageously water or a water/glycerol mixture.

According to a particular mode, the extract is an aqueous extract, in particular a hydro glycerin extract.

Advantageously, it is an extract obtained by aqueous extraction and is therefore an aqueous extract of Opuntia ficus-indica prickly pear cladodes.

Advantageously, the extract of prickly pear, Opuntia ficus-indica, is an extract obtained via a solid/liquid extraction method, in particular an aqueous extract or a hydro-glycerin extract. Even more advantageously, it is a prickly pear stem (or cladode) extract.

Extraction is performed using conventional solid/liquid extraction techniques, by bringing the solid material to be extracted into contact with the extraction solvent, followed by a solid/liquid separation step to recover the liquid phase loaded with the compounds solubilized in this liquid phase.

Extraction can be carried out with agitation or statically, at room temperature or with heating. It can be assisted by ultrasound, microwave, flash détente or extrusion, in a plant weight/volume solvent ratio that can vary from 1/3 to 1/30, for a period of 1 minute to 48 hours. Extraction may be repeated 2 to 3 times.

Extraction can be assisted by the use of at least one enzyme selected from pectinases, cellulases (endo or exocellulases), hemicellulases, beta-glucanases and xylanases, as well as mixtures thereof.

The solid fraction (also known as pomace) can then be separated from the liquid extract by any suitable means, such as centrifugation, decantation or filtration, to recover a clear, particle-free liquid phase. The liquid phase representing the extract can be more or less concentrated, or even dried, until a dry extract is obtained.

A particularly advantageous extract for uses according to the present invention is an extract of prickly pear Opuntia ficus-indica obtained via a solid/liquid extraction method, in particular an aqueous extract of prickly pear Opuntia ficus-indica containing, in % by weight, from 40 to 60% water, from 40 to 60% glycerol, from 0 to 2% 2-phenoxyethanol and from 0.5 to 8% dry matter of prickly pear Opuntia ficus-indica stem extract.

Preferably such a prickly pear Opuntia ficus-indica extract obtained via a solid/liquid extraction method, in particular an aqueous prickly pear Opuntia ficus-indica extract, contains, in % by weight, 2.5% to 5% dry matter aqueous prickly pear Opuntia ficus-indica extract, 45 to 50% glycerol, 0 to 1% 2-phenoxyethanol and 45 to 50% water.

A particularly suitable extract is also commercially available under the name AquaCacteen® or AquaCacteen® nc from MIBELLE AG BIOCHEMISTRY (CH).

### Cosmetic Compositions

The present invention also relates to a cosmetic composition comprising a prickly pear extract as described above and at least one cosmetically acceptable excipient, particularly suitable for topical application and/or oral administration, preferably for topical application.

The invention is preferably aimed at cosmetic compositions in a form suitable for topical application, preferably directly to keratin fibers, advantageously to the hair.

Cosmetic compositions according to the invention can thus be presented in the forms that are usually known for topical administration, i.e., in particular lotions, shampoos, balms, foams, gels, dispersions, emulsions, sprays, serums, masks or creams, with excipients notably enabling better penetration into the skin appendages in order to improve the properties and accessibility of the active ingredient/agent.

Advantageously, the cosmetic compositions according to the invention can be in the form of hair compositions. Such compositions are usually known for topical application to the hair, i.e., in particular a shampoo, conditioner, hair cream, hair lotion, mask or leave-on spray.

Preferably, the compositions according to the invention are said to be neutral and gentle on the hair fiber.

A distinction is thus made between formulated products that can be rinsed and formulated products that do not require rinsing.

Advantageously, the compositions according to the invention are applied to keratin fibers, preferably the hair, without rinsing or for at least 5 minutes before rinsing.

These compositions generally contain, in addition to the prickly pear extract described above, a physiologically acceptable medium, generally water- or solvent-based, for example alcohols, ethers or glycols. They can also contain surfactants, complexing agents, preservatives, stabilizers, emulsifiers, thickeners, gelling agents, humectants, emollients, trace elements, essential oils, fragrances, colorants or thermal waters, etc.

In some embodiments of the invention, the extract useful in the context of the invention is the only active agent used to tighten the scales of the composition. It is also typically the only active agent in the cosmetic composition that moisturizes, cares for, strengthens and/or protects the skin appendages, in particular keratin fibers, and even more particularly the hair.

Advantageously, the cosmetic compositions according to the present invention will comprise 0.01 to 5% by weight of dry extract, preferably 0.02 to 1% by weight, even more preferably 0.05 to 0.5% by weight of prickly pear dry extract relative to the total weight of the composition. Preferably, the composition comprises 0.1% by weight of prickly pear solids, based on the total weight of the composition. These % by weight of dry extract exclude the weight of any drying support, if any, and relate only to the dry plant extract.

The present invention also relates to a hair composition comprising as active principle (active agent) a hair treatment agent comprising or consisting of a prickly pear extract as described in the present description, and a hair-acceptable excipient.

According to an advantageous embodiment, the hair composition according to the invention is characterized in that it is in a form suitable for topical administration at the level of the skin appendages, in particular at the level of the keratin fibers, even more particularly at the level of the hair.

In the present invention, “capillary acceptable” is taken to mean that which is useful in the preparation of a hair composition, which is generally safe, non-toxic and neither biologically nor otherwise undesirable and which is acceptable for use by topical application to the skin appendages, in particular to keratin fibers, still more particularly to the hair.

Cosmetic compositions can be manufactured using methods well known to those skilled in the art.

The invention also relates to a method of preparing a hair composition for topical application to keratin fibers, more particularly the hair; the composition comprising a prickly pear extract, the method comprising the steps of: a) mixing the prickly pear extract as described above with a dermatologically or cosmetically acceptable vehicle; and b) packaging the mixture resulting from step (a) in a suitable package.

Uses and Methods

Prickly pear extract as described above or the cosmetic composition according to the present invention is useful for tightening the scales of keratin fibers, in particular hair. By tightening the scales, the prickly pear extract useful in the context of the invention or the cosmetic composition provides a protective and repairing effect on the surface of the hair fiber.

The prickly pear extract useful within the framework of the invention or the cosmetic composition according to the present invention furthermore makes it possible to moisturize and/or protect the skin appendage, preferably the keratinous fibers, preferably the head hair and/or the body hair, even more preferably the head hair.

More particularly, the prickly pear extract useful in the context of the invention or the cosmetic composition according to the invention also makes it possible to protect the structure, visual properties and/or biomechanical properties of keratin fibers against mechanical, biological, chemical and/or environmental aggressions.

The cosmetic use of a prickly pear extract as described above or of a cosmetic composition comprising such an extract also makes it possible to prevent and/or reduce and/or eliminate unsightly disorders caused by chemical, biological, physical, mechanical and/or environmental aggression on the skin appendages, preferably keratin fibers, and even more preferably the hair, preferably to maintain and/or increase the softness and/or silkiness of the keratin fibers, and/or to beautify the keratin fibers, in particular to make them shinier and/or improve their shine, and/or to improve and/or facilitate their shaping and/or styling, thereby maintaining and/or increasing their resistance, vitality and/or strength.

Advantageously, the protection of keratin fibers, in particular hair, involves protecting these fibers when they are subjected to physical or mechanical aggression, alteration, degradation, damage or stress. In particular, these alterations, degradations, damage or mechanical constraints can be chosen from the group comprising brushing, straightening, drying, pulling, combing and curling, either individually or in combination.

Advantageously, the protection of keratin fibers, in particular hair, involves protecting these fibers when they are subjected to chemical aggressions selected from dyeing and/or bleaching.

The protection of skin appendages, and in particular keratin fibers, can be assessed using existing techniques in the field. Conventional methods for measuring a protection effect measure the ability of the product under evaluation to restore a visual, structural and/or functional state of a damaged skin appendage, preferably a damaged keratinous fiber, comparable to the state of an undamaged skin appendage, preferably an undamaged keratinous fiber. They are applied to skin appendages, preferably to keratin fibers, even more preferably to damaged hair, and the protective effect is measured by comparison with skin appendages, preferably keratin fibers, even more preferably undamaged hair.

Prickly pear extract useful in the context of the invention, or the cosmetic composition according to the invention, is particularly suitable for the care and/or treatment of damaged keratin fibers and/or keratin fibers exposed to physical, mechanical and/or biological and/or chemical and/or environmental aggressions, in particular damaged hair, especially hair that is dry, split, dull, fine, colored, rough, flat, lackluster and/or difficult to style and/or shape.

The prickly pear extract used in the invention, or the cosmetic composition used in the invention, is also suitable for the care of hair which, by its very nature, becomes fragile and rapidly damaged, such as dry and/or curly and/or frizzy and/or white and/or ageing and/or fine hair.

Prickly pear extract useful in the context of the invention or the cosmetic composition according to the invention is also particularly suitable for use on the hair, in particular for maintaining and/or increasing the strength and vitality of the hair and/or for strengthening the hair.

Prickly pear extract useful in the context of the invention or the cosmetic composition according to the invention is also particularly suitable for use on the hair, in particular to maintain and/or increase shine and/or luster, softness and/or silkiness, thereby facilitating styling and/or shaping of the hair.

Prickly pear extract useful within the scope of the invention, or the cosmetic composition according to the invention, can be applied to all or part of the skin appendages, preferably the keratinous fibers of the human body selected from head hair, nails and body hair, in particular head hair and body hair, especially beard hair, eyelashes and/or eyebrows, advantageously hair.

Prickly pear extract useful in the context of the invention, or the cosmetic composition according to the invention are particularly useful for dry hair, damaged hair, in particular dull, fine, colored, rough, flat, lackluster and/or difficult to style and/or shape and/or hair which becomes fragile and/or rapidly damaged due to its nature such as dry and/or curly and/or frizzy and/or white and/or aging and/or fine hair.

The invention also covers the use of an active ingredient comprising, or consisting of, a prickly pear extract to protect and/or repair the skin appendages.

The present invention also relates to a cosmetic care method characterized in that it comprises the application to a concerned area of the skin appendages, preferably of the keratin fibers, of the face and/or body, of the prickly pear extract useful within the framework of the invention or of the cosmetic composition according to the invention, to moisturize and/or protect the skin appendages and in particular to maintain and/or increase shine and/or luster, softness and/or silkiness, strength, vitality and/or tighten the hair scales and/or strengthen the hair and thus facilitate styling and/or shaping of the hair.

The invention is thus aimed at a cosmetic method for moisturizing and/or protecting skin appendages comprising the topical application of a prickly pear extract as described above or of a cosmetic composition according to the invention to a concentrated area of the skin appendages.

In particular, the skin appendages are chosen from the keratin fibers of the face and/or body. Advantageously, keratin fibers are chosen from the group consisting of hair, eyelashes, bristles and nails. Even more particularly, they are hair.

The present invention thus relates to the use of a hair treatment agent comprising or consisting of a prickly pear extract, obtained by solid/liquid extraction, in particular an aqueous or hydro-glycerin prickly pear extract, for moisturizing, caring for, strengthening and/or protecting the skin appendages, in particular keratin fibers, even more particularly the hair.

The invention also covers the use of a hair treatment agent comprising or consisting of a prickly pear extract, obtained by solid/liquid extraction, in particular an aqueous or hydro-glycerin prickly pear extract, for moisturizing skin appendages, in particular keratin fibers, and even more particularly hair.

Advantageously, the moisturizing of keratin fibers, in particular hair, involves moisturizing these fibers when they are subjected to physical or mechanical aggression, alteration, degradation, damage or stress. In particular, these alterations, degradations, damage or mechanical constraints can be chosen from the group comprising brushing, straightening, drying, pulling, combing and curling, either individually or in combination.

Advantageously, the protection of keratin fibers, in particular hair, involves protecting these fibers when they are subjected to physical or mechanical aggression, alteration, degradation, damage or stress. In particular, these alterations, degradations, damage or mechanical stresses can be selected from the group comprising brushing, straightening, drying, pulling, combing and curling, either individually or in combination.

Equally advantageous, the moisturizing of keratin fibers, in particular hair, involves moisturizing these fibers when they are subjected to chemical aggressions selected from dyeing and/or bleaching.

Equally advantageously, the protection of keratin fibers, in particular hair, involves protecting these fibers when they are subjected to chemical aggressions selected from dyeing and/or bleaching.

The present invention also relates to a method for tightening the scales of keratin fibers or for obtaining moisturization, care, reinforcement and/or protection of skin appendages, in particular keratin fibers, still more particularly hair, comprising the steps of: (i) wetting the skin appendages, in particular the keratin fibers, more particularly the hair, with water; (ii) applying an effective amount of a cosmetic composition according to the present invention to the skin appendages, in particular the keratin fibers, more particularly the hair; (iii) optionally rinsing the composition with water; and (iv) optionally repeating steps (ii) and (iii).

The inventors have thus demonstrated that a hair treatment agent comprising, or consisting of, a prickly pear extract, obtained by solid/liquid extraction, in particular an aqueous or hydro-glycerin prickly pear extract, can advantageously be used in a hair composition intended for application directly to the skin appendages, in particular the keratin fibers, more particularly the hair, to moisturize, care for, strengthen and/or protect it.

The term “skin appendage care, in particular for keratin fibers, and even more specifically for hair” refers to the enhancement of their integrity and/or visual properties, such as softness and/or silkiness, shine and/or luster, and/or biomechanical properties, such as resistance, vitality and/or strength, and in particular for hair, to facilitate styling and/or shaping.

The invention also relates to a method of cosmetic treatment of skin appendages, in particular keratin fibers, even more particularly hair, with a hair composition according to the invention and as defined in the present description.

The invention also relates to the use of a hair treatment agent comprising or consisting of a prickly pear extract obtained by solid/liquid extraction, in particular an aqueous or hydro-glycerin prickly pear extract, for the manufacture of a hair composition intended for moisturizing, caring for, strengthening and/or protecting skin appendages, in particular keratin fibers, even more particularly hair. The following examples illustrate the invention without limiting its scope.

### EXAMPLES

### Example 1: Low-Field NMR Evaluation of the Moisturizing Effect of Prickly Pear Extract on Hair

Nuclear Magnetic Resonance (NMR) methods can be used to characterize the moisturizing capacity of molecules such as an extract according to the invention, either directly in water or in contact with hair.

The aim of this study was to investigate, using low-field NMR methods, the possible impact of the addition of prickly pear extract on hair moisturizing capacity.

Prickly pear extract was taken in distilled water at a concentration of 2%.

The commercial name of this material is an extract sold under the brand name Aquacacteen® nc by the company Mibelle Biochemistry (Switzerland), containing in % by weight, 45 to 50% glycerol, 0 to 1% 2-phenoxyethanol and 45 to 50% water and 5% by weight dry prickly pear extract; this amounts to a tested concentration of 0.1% dry extract. Examples 1 to 4 use this material.

A positive control, known as a moisturizer, is chosen to validate the test; this is glycerin, also tested at 2% by weight.

Before analysis, the hair strands were prepared as follows:
Moisten strands under cold running water,
Wash strands with shampoo (extra-gentle from Ducray®), 1.5 mL of shampoo per strand,
Rinse with cold water,
Blot and blow-dry on cold setting for 3 minutes,
Lay hair back to dry,
Soak strands in distilled water solution or prickly pear extract for 1 hour,
After one hour, remove the strands,
Comb the strands,
Leave to dry at room temperature for 1 hour,
After one hour, blow-dry on cold for 2 minutes,
Cut 2 cm from tips,
Cut 6 cm and insert into NMR tube,
Allow samples to equilibrate for 24 hours at +4° C.,
Analyze.

### Humidity Measurement

For each condition, 2 humidity measurements were carried out. Water content was determined by differential weighing after 35 minutes of steaming at 135° C.

### Low-Field NMR Analysis

For each condition, 3 series of low-field NMR measurements were performed on a Brucker Minispec mq20 operating at 20 MHz. The various measurements carried out enabled us to evaluate information relating to:
The total number of protons present in the sample (essentially the water signal),
Mobility of protons, representative of the bonding forces of water,
Proton compartmentalization within the product (highly bound/highly free protons).

Hydration water is called highly bound water, as it is strongly interacted with by macromolecules (melanin and keratin). Another population, called bound water, is indirectly influenced by macromolecules, and “very free” water is not subject to any interaction. Within the system, free water is more or less mobile. This mobility is measured and is representative of the bonding forces of water. The lower the mobility, the stronger the bonding forces between water and macromolecules.

### Results

Humidity content results are summarized in Table 1 below:

TABLE 1
Humidity content measured in analyzed hair strands

| Groups | % (mean ± SD) |
|---|---|
| Untreated | 10.13 ± 0.53 |
| Distilled water | 9.98 ± 0.10 |
| Glycerin | 9.15 ± 0.23 |
| Extract according to invention | 9.87 ± 0.05 |

Avg: mean;
SD: standard deviation

Compared to untreated hair, the application of distilled water, glycerin or prickly pear extract does not increase humidity content.

### Low-Field NMR Analysis

Table 2 shows the proton mobility measured by low-field NMR in the different hair strands analyzed. This parameter is representative of the bonding forces between water and the constituent molecules of the extract according to the invention or its excipient and the hair. The lower the proton mobility, the stronger the bonding forces, and vice versa.

Table 2: Average mobility of protons in the different strands analyzed

| Groups | Mobility of protons (ms) |
|---|---|
| Untreated | 3.10 ± 1.01 |
| Distilled water | 2.87 ± 0.46 |
| Glycerin | 3.23 ± 1.10 |
| Extract according to invention | 2.43 ± 0.25 |

Compared to untreated hair, the application of distilled water alone tends to decrease the mobility of protons very slightly, to be linked to weak bonding forces between water and hair. Application of glycerin does not induce any significant change in the mobility of protons. Compared to hair treated with distilled water, prickly pear extract leads to a decrease in the mobility of protons in the hair. This marked change reflects an increase in bonding forces.

Table 3 shows the total number of protons visible by low-field NMR in the different strands analyzed.

TABLE 3
Total number of protons visible by low-field NMR

| Groups | Number of protons (UA/g) |
|---|---|
| Untreated | 349.6 ± 10.8 |
| Distilled water | 366.7 ± 3.8 |
| Glycerin | 375.8 ± 9.9 |
| Extract according to invention | 384.7 ± 5.0 |

Compared to untreated hair, the application of distilled water alone moderately increases the total amount of water visible by low-field NMR in the strands, reflecting hair moisturizing. Glycerin also increases the number of protons. This slightly greater increase than that found with distilled water validates this test, highlighting the moisturizing effect of glycerin. Compared to hair treated with distilled water, prickly pear extract results in a marked increase in the total amount of water in the strands, which is associated with better hair moisturizing.

This total amount of water visible by low-field NMR dissociates into two populations, highly bound water and bound water.

Table 4 shows the numbers of highly bound protons and bound protons measured by low-field NMR in the different hair strands analyzed.

TABLE 4
Numbers of highly bound protons and bound
protons measured by low-field NMR

| | Number of bound | Number of highly bound |
|---|---|---|
| Groups | protons (UA/g) | protons (UA/g) |
| Untreated | 41.4 ± 7.6 | 308.2 ± 8.0 |
| Distilled water | 45.2 ± 5.0 | 321.5 ± 8.8 |
| Glycerin | 41.4 ± 9.0 | 334.4 ± 0.9 |
| Extract according | 54.4 ± 5.0 | 330.3 ± 2.8 |
| to invention | | |

Overall, the evolution of these two parameters is identical to that of the total amount of water.

In fact, the application of distilled water slightly increases the number of bound or highly bound protons. Compared with hair treated with distilled water, prickly pear extract increases the number of bound and highly bound protons. Glycerin only increases the number of highly bound protons.

In conclusion, this technique was validated by the results found with the well-known moisturizing agent glycerin. The technique demonstrated that prickly pear extract induces a moisturizing effect on strands of normal hair. In moisturized hair, the total amount of water visible by low-field NMR is increased compared to non-moisturized hair. This water, which is essentially highly bound water, is representative of the hair's moisturizing capacity. In fact, the greater the total water content of a hair, the higher its amount of highly bound water, and the more moisturized it will be. At the same time, the mobility of protons will be representative of bonding forces. In this context, prickly pear extract is highly effective, providing excellent moisturizing capacity with strong bonding forces.

### Example 2: Low-Field NMR Evaluation of the Moisturizing Effect of Prickly Pear Extract on Damaged Hair

The aim of this study is to reproduce the study of Example 1 on damaged hair, with and without rinsing after application of prickly pear extract.

The prickly pear extract was taken in distilled water at a concentration of 2%. This is the same extract as in Example 1.

Several groups are performed on damaged hair, with two strands per group:
An untreated group
A group with distilled water only
A group with 2% glycerin (positive control)
A group with prickly pear extract
The latter subdivided into 2 groups with and without rinsing.

The protocol used is the same as in Example 1. Before combing, the strands are rinsed under cold water for 30 seconds.

### Protocol for Damaged Hair:

Natural dark brown 5/0 virgin hair (supplier Kerling International, Germany) was bleached using a commercial preparation. The preparation consisted of a persulfate-based powder mixed with 12-volume oxygenated water, forming a paste which was spread onto strands of hair, then wrapped in aluminum foil. After 30 minutes, the hair strands were rinsed thoroughly and dried. This bleaching procedure was repeated on the same strands to obtain hair treated for a total of 2×30 minutes.

### Results

The impact of the bleach treatment (unrinsed normal hair versus unrinsed damaged hair) is summarized in Table 5.

TABLE 5
Bleaching effect

| Parameters | Normal hair | Damaged hair |
|---|---|---|
| Humidity content (%) | 10.13 ± 0.53 | 11.19 ± 0.30 |
| Mobility of protons (ms) | 3.10 ± 1.01 | 2.63 ± 0.25 |
| Number of protons (UA/g) | 349.6 ± 10.8 | 409.1 ± 4.5 |
| Number of bound protons (UA/g) | 41.4 ± 7.6 | 39.6 ± 2.7 |
| Number of highly bound protons (UA/g) | 308.2 ± 8.0 | 369.5 ± 7.2 |

Bleaching causes variations in all the parameters evaluated. These include an increase in the water content of the hair, a decrease in the mobility of protons and a sharp rise in the number of protons, due to an increase in the number of highly bound protons.

The moisturizing effect of prickly pear extract applied to damaged hair is summarized in Table 6 below:

TABLE 6
Moisturizing effect of prickly pear extract

| | | | Extract |
|---|---|---|---|
| | Distilled | | according to |
| Parameters | water | Glycerin | the invention |
| Humidity content (%) | 11.19 ± 0.09 | 12.00 ± 0.05 | 12.02 ± 0.10 |
| Mobility of protons (ms) | 2.73 ± 0.15 | 2.53 ± 0.12 | 1.97 ± 0.50 |
| Number of protons (UA/g) | 416.6 ± 5.6 | 427.6 ± 16.5 | 430.8 ± 9.6 |
| Number of bound protons | 39.2 ± 4.1 | 38.6 ± 1.7 | 51.5 ± 12.0 |
| (UA/g) | | | |
| Number of highly bound | 377.4 ± 7.7 | 388.9 ± 18.0 | 379.3 ± 11.4 |
| protons (UA/g) | | | |

Compared to untreated damaged hair, the application of water alone does not alter the humidity content of the strands or the mobility of the protons making up damaged hair.

The application of glycerin induces an increase in humidity content, associated with a decrease in the mobility of protons.

Compared to damaged hair treated with water, the application of prickly pear extract resulted in a marked increase in humidity content and an equally marked decrease in the mobility of protons in damaged hair. This evolution reflects an increase in bonding forces.

Compared to untreated damaged hair, the application of distilled water alone increases very slightly the total number of protons visible by low-field NMR in the strands.

As expected, glycerin, a known hair moisturizing agent, induced an increase in the total number of protons.

Compared to damaged hair treated with water, the application of prickly pear extract will lead to an increase in the total number of protons, to be associated with better hair moisturizing. This increase is most apparent in the number of bound protons.

The aim of this study, which was to assess whether the moisturizing effects of a prickly pear extract demonstrated on normal hair could be reproduced on damaged hair, has been perfectly achieved.

Finally, the impact of a water rinse on damaged hair was evaluated. Its effects are summarized in Table 7 below:

TABLE 7
Impact of rinsing on damaged hair

| | | Extract according |
|---|---|---|
| Parameters | Glycerin | to the invention |
| Humidity content (%) | 11.84 ± 0.26 | 12.30 ± 0.01 |
| Mobility of protons (ms) | 3.00 ± 0.36 | 1.77 ± 0.25 |
| Number of protons (UA/g) | 416.9 ± 3.4 | 443.8 ± 5.2 |
| Number of bound protons (UA/g) | 31.7 ± 0.3 | 63.0 ± 10.1 |
| Number of highly bound protons | 385.2 ± 3.5 | 380.7 ± 4.9 |
| (UA/g) | | |

Depending on the active ingredient, the rinsing step does not have the same impact on damaged hair strands: with glycerin, the water content does not vary, whereas in the presence of prickly pear extract, the humidity content increases slightly.

The same applies to the mobility of protons: with glycerin, the mobility of protons increases, leading to a reduction in bonding forces, whereas with prickly pear extract, the mobility of protons remains unchanged.

The number of protons does not change significantly for either group.

On the other hand, the rinsing step does not have the same impact on the number of bound protons: with glycerin, a decrease in this number is observed, whereas in the presence of prickly pear extract, the number of bound protons is increased.

The number of highly bound protons does not appear to be affected in either group.

The inventors clearly demonstrate that prickly pear extract has moisturizing properties not only on normal hair, but also on damaged hair, and that the water penetrates on rinsing. In fact, the moisturizing results appear to be better than those obtained with the well-known moisturizing agent glycerin, which loses its effectiveness on rinsing.

### Example 3: Low-Field NMR Evaluation of the Moisturizing Effect of Prickly Pear Extract on Damaged Hair

The aim of this study is to reproduce the study in Example 2, again on damaged hair, with and without rinsing after application of a prickly pear extract. The persistence effect was also tested.

Prickly pear extract was taken in distilled water at concentrations of 0.5 and 1%.

This is the same extract as in Example 1, but 2 and 4 times less concentrated.

Several groups were performed on damaged hair, with two strands per group:
An untreated group
A group with distilled water only
A group with 0.5% prickly pear extract
A group with 1% prickly pear extract

These last two groups are subdivided into 2 with and without rinsing. The rinse step, which takes place before combing, involves rinsing the strands under cold water for 30 seconds.

For the no-rinse group, an analysis is carried out at D0, 24 hours and 48 hours to assess residual effects. The protocol for damaged hair is the same as in example 2.

### Results

The moisturizing effect of 0.5% or 1% prickly pear extract applied to damaged, unrinsed hair is summarized in Table 8 below:

TABLE 8
Moisturizing effect of prickly pear extract

| | | | Extract | Extract |
|---|---|---|---|---|
| | | | according | according |
| | | | to the | to the |
| | | Distilled | invention | invention |
| | Untreated | water | 0.5% | 1% |
| Humidity | 11.63 ± 0.40 | 11.61 ± 0.11 | 11.28 ± 0.15 | 11.88 ± 0.79 |
| content (%) | | | | |
| Mobility of | 1.30 ± 0.20 | 1.27 ± 0.32 | 1.30 ± 0.10 | 0.83 ± 0.11 |
| protons (ms) | | | | |
| Total number | 385.4 ± 14.6 | 382.1 ± 7.2 | 391.1 ± 17.7 | 405.3 ± 13.9 |
| of protons | | | | |
| (UA/g) | | | | |
| Number of | 61.3 ± 12.3 | 57.1 ± 10.6 | 58.9 ± 7.5 | 125.3 ± 20.4 |
| bound protons | | | | |
| (UA/g) | | | | |
| Number of | 324.1 ± 15.3 | 325.0 ± 17.7 | 332.2 ± 24.4 | 280.0 ± 23.4 |
| highly bound | | | | |
| protons (UA/g) | | | | |

Humidity content of damaged hair is broadly identical in all groups.

Compared to damaged hair treated with water, the application of a 1% prickly pear extract resulted in a marked decrease in the mobility of protons. This reflects an increase in the bonding forces between water and the extract's constituent molecules.

Compared to damaged hair treated with distilled water alone, the application of 0.5% prickly pear extract tends to increase the total number of protons visible by low-field NMR in the strands, and when applied at 1%, increases the total number of protons more significantly. This effect is linked to better hair moisturization with 1% prickly pear extract.

In the presence of 1% prickly pear extract, the number of bound protons is greatly increased.

The inventors have clearly demonstrated that prickly pear extract, even at a low concentration, is particularly effective in moisturizing damaged hair.

A study of the persistence of the moisturizing activity of a 1% prickly pear extract applied to damaged, unrinsed hair is summarized in Table 9 below:

TABLE 9
Persistence of moisturizing activity of prickly pear extract

| | | Extract | Extract | Extract |
|---|---|---|---|---|
| | | according | according | according |
| | Distilled | to the | to the | to the |
| | water, | invention | invention | invention |
| | J0 | 1%, J0 | 1%, 24 h | 1%, 48 h |
| Mobility of protons (ms) | 1.27 ± 0.32 | 0.83 ± 0.11 | 0.97 ± 0.07 | 1.23 ± 0.06 |
| Total number of protons | 382.1 ± 7.2 | 405.3 ± 13.9 | 406.7 ± 9.5 | 387.5 ± 2.7 |
| (UA/g) | | | | |
| Number of bound protons | 57.1 ± 10.6 | 125.3 ± 20.4 | 102.5 ± 1.7 | 72.7 ± 5.2 |
| (UA/g) | | | | |

These results show that the moisturizing effect of prickly pear extract on damaged, unrinsed hair lasts for at least 24 hours. The mobility of protons is reduced at 24 hours in the group treated with 1% prickly pear extract, while the total number of protons remains high compared with the application of distilled water, and the number of bound protons appears higher even 48 hours after the application of 1% prickly pear extract.

The inventors have thus demonstrated the persistence of prickly pear extract for 24 hours on unrinsed hair.

### Example 4: Observation of Hair Using Scanning Electron Microscopy

### Hair Preparation for Microscopy

5-6 damaged hairs (same protocol as in example 2) are mounted on aluminum bases using carbon adhesive disks. For each hair, a knot is tied to apply stress to the hair. This stress highlights the cuticle scales which lack cohesion, visible by their lifting. On the day of analysis, these sockets are placed in the scanning electron microscope chamber.

The surface of the hair is observed, with photographs taken at a magnification of between×700-1000, before and after the application of prickly pear extract.

Prickly pear extract is tested at 1%.

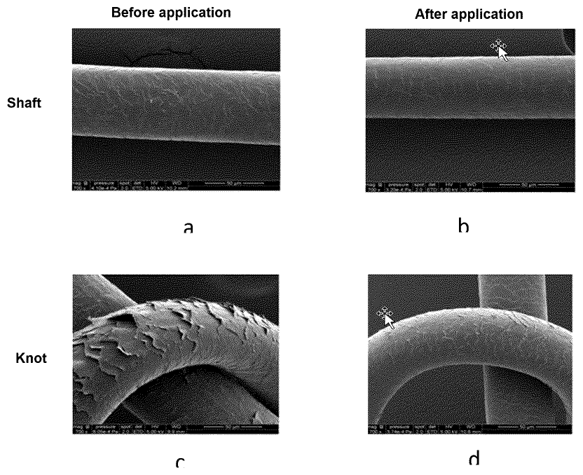

The results are shown in FIG. 1 ((a) shaft before application; (b) shaft after application; (c) knot before application; (d) knot after application).

It appears that prickly pear extract tested at 1% tightens hair scales, providing a protective and repairing effect on the surface of the hair fiber.

1. A cosmetic method to tighten the scales of keratin fibers comprising the step of topically applying a prickly pear extract to keratin fibers.

2. The cosmetic method according to claim 1, characterized in that the prickly pear extract is an aqueous or hydro-glycerine extract of prickly pear.

3. The cosmetic method according to claim 1, characterized in that the prickly pear extract is an extract of prickly pear stems.

4. The cosmetic method according to claim 1 that further protects the structure, visual properties and/or biomechanical properties of keratin fibers against mechanical, biological, chemical and/or environmental aggression.

5. The cosmetic method according to claim 1 wherein keratin fibers are hair.

6. The cosmetic method according to claim 1 wherein keratin fibers are dry hair.

7. The cosmetic method according to claim 1 wherein keratin fibers are damaged hair.

8. The cosmetic method according to claim 1 to that maintain and/or increase hair vitality.

9. (canceled)

10. A cosmetic method for tightening the scales of keratin fibers comprising the step of topically applying a composition comprising a prickly pear extract and at least one cosmetically acceptable excipient to keratin fibers.

11. The cosmetic method according to claim 10, characterized in that the prickly pear extract is an aqueous or hydro-glycerine extract of prickly pear.

12. The cosmetic method according to claim 10, characterized in that the cosmetic composition comprises 0.01 to 5% by weight of dry prickly pear extract relative to the total weight of the composition.

13. The cosmetic method according to 10 to further protects the structure, visual properties and/or biomechanical properties of keratin fibers against mechanical, biological, chemical and/or environmental aggressions.

14. (canceled)

15. The cosmetic method according to 10, wherein keratin fibers are hair.

16. The cosmetic method according to claim 10, wherein keratin fibers are dry hair.

17. The cosmetic method according to claim 10, wherein keratin fibers are damaged hair.

18. The cosmetic method according to claim 10, that further maintain and/or increase hair vitality.

19. The cosmetic method according to claim 1, wherein keratin fibers are dull, fine, color-treated, rough, flat or lacks shine hair.

20. The cosmetic method according to claim 1, wherein keratin fibers are hair difficult to style and/or shape.

21. The cosmetic method according to claim 1, wherein keratin fibers are hair that are fragile and/or rapidly damaged due to its very nature.

22. The use according to claim 1, wherein keratin fibers are dry and/or curly and/or frizzy and/or white and/or ageing and/or fine hair.

23. The cosmetic method according to claim 10, wherein keratin fibers are dull, fine, color-treated, rough, flat or lacks shine hair.

24. The cosmetic method according to claim 10, wherein keratin fibers are difficult to style and/or shape.

25. The cosmetic method according to claim 10, wherein keratin fibers are hair that are fragile and/or rapidly damaged due to its very nature.

26. The use according to claim 10, wherein keratin fibers are dry and/or curly and/or frizzy and/or white and/or ageing and/or fine hair.
